(19) United States
(12) Patent Application Publication
(10) Pub. No.: US 2022/0115094 A1
(43) Pub. Date: Apr. 14, 2022

(54) FREE ENERGY PERTURBATION COMPUTATION SCHEDULING METHOD USED IN HETEROGENEOUS CLUSTER ENVIRONMENT

(71) Applicant: SHENZHEN JINGTAI TECHNOLOGY CO., LTD., Guangdong (CN)

(72) Inventors: Zenghui LIU, Guangdong (CN); Mi HE, Guangdong (CN); Mingjun YANG, Guangdong (CN); Lipeng LAI, Guangdong (CN); Jian MA, Guangdong (CN); Shuhao WEN, Guangdong (CN)

(73) Assignee: SHENZHEN JINGTAI TECHNOLOGY CO., LTD., Guangdong (CN)

(21) Appl. No.: 17/266,115

(22) PCT Filed: Feb. 25, 2020
(86) PCT No.: PCT/CN2020/076599

§ 371 Date: Feb. 5, 2021

(51) Int. Cl. G16C 20/50; G16C 10/00

(52) U.S. Cl. CPC G16C 20/50; G16C 10/00

(57) ABSTRACT

The invention provides a free energy perturbation computation scheduling method used in a heterogeneous cluster environment, including the following steps. Step A: performing npt ensemble dynamic simulations through pre-built molecular/protein structures and input files to obtain equilibrium structures; Step B: running replica exchange dynamic calculations based on Hamiltonian to obtain enough trajectory data; Step C: analyzing trajectory files, and combining the trajectory files with various prmtop to generate new amber calculation inputs, calculating a single point energy corresponding to each conformation after combination; Step D: using regular expressions to extract energy values from log files, and at the same time cleaning up intermediate temporary files to complete a calculation process of a single molecule.

### BACKGROUND

### Technical Field

The invention is related to the technical field of high-performance computing and drug design, and specifically is a free energy perturbation computation scheduling method used in a heterogeneous cluster environment: used in a heterogeneous (cpu+gpu) architecture cluster to provide efficient resource utilization and micro-scheduling requirements for free energy perturbation computing services.

### Description of Related Art

The update of modern computer hardware GPU has made the graphics card GPU has powerful data parallel computing capabilities. Combining CPU and GPU to build heterogeneous clusters can easily obtain powerful computing capabilities, which are particularly suitable for computing-intensive applications. More and more high-performance computing (HPC) users are migrating to GPU-based clusters, so as to run their scientific and engineering applications. In a heterogeneous computing environment, users are allowed to use both CPU and GPU in the calculation model at the same time, wherein the continuous part of the application runs on the CPU and the computationally intensive part runs on the GPU. Compared with the traditional CPU-based model, the speed that users run applications is greatly improved by tapping the massive parallel capability of GPU.

Among various drug design methods, free energy perturbation (FEP) is a high-precision method for evaluating the binding strength of small drug molecules and targets. It can effectively remove false positive molecules, increase the design success rate, and accelerate the development of new drugs. The FEP process based on enhanced sampling algorithm, high-precision molecular force field (Xforce) and rigorous data statistical analysis method needs to accurately calculate the binding free energy of hundreds of small molecule drug candidate compounds and targets in a short time. XFEP's prediction error (mean unsigned error) of functional group replacement and skeleton transition calculation in dozens of test systems and a series of actual projects are all less than 1.0 kcal/mol, and the predicted value and the data obtained in the experiment show a significant correlation. This high-precision method requires support from a large amount of calculation, and industrial applications require timeliness of the computing system. Therefore, it is necessary for the calculation time and accuracy to be improved as much as possible.

### SUMMARY

In order to solve the above technical problems, the invention provides a free energy perturbation computation scheduling method used in a heterogeneous cluster environment, which makes full use of gpu+cpu heterogeneous cluster resources to undertake mass free energy perturbation calculations, especially in the use of replica exchange based on the temperature of the solvent (a replica exchange method assuming that when the temperature rises, the solvent molecules move while the solute molecules are locked).

Free energy perturbation is a common method for calculating free energy. Taking the canonical ensemble as an example, the free energy change from state a to state b can be calculated by the following formula:

$$Δ F ( A \to B ) = {F}_{B} - {F}_{A} = - {k}_{B} T ln {〈 exp ( - \frac{{H}_{B} - {H}_{A}}{{k}_{B} T} ) 〉}_{A}$$

wherein T is the temperature, H_{A} and H_{B} are Hamiltonians of state A and state A respectively; kB is Boltzmann's constant, and < > represents the ensemble average in the state A. In other words, in order to calculate the free energy difference between state A and state B, it is necessary to sample the energy difference between the two states in the ensemble of state A and then average. Sampling can be simulated using molecular dynamics or Monte Carlo method. Since accurate free energy calculation requires a large amount of sampling, the amount of calculation brought by it is very large. In the implementation process, in order to speed up the sampling process, a series of enhanced sampling methods have been developed, among which Replica Exchange with Solute Tempering (rest2) is one of the effective enhanced sampling methods. The Rest2 method is based on the replica exchange dynamics of the Hamiltonian. By enhancing the sampling of neighboring replicas, the freedom degree of interest can be selected and the biasing intensity that is related to (exp (−V/kt)) speeds up the free energy calculation process.

The specific steps included are as follows:

A free energy perturbation computation scheduling method used in a heterogeneous cluster environment includes the following steps:
characterized in that, it is applied to a cluster scheduler in a heterogeneous cluster, the heterogeneous cluster includes many nodes, and the method includes:
taking the different calculation characteristics of the task and the characteristic parameters of the calculation node; the calculation characteristics of the task include: running npt ensemble balance simulation, running remd parallel calculation and checking out whether it needs GPU support, single point energy calculation of the structure, and the analysis and processing of a large number of small files, etc.; the characteristics of a computing node include: the number of free CPUs of the node, memory usage, and the utilization of GPUs;

step A: In the header directory, using a recursive algorithm to scan multiple folders, scanning structures and files such as molecules/proteins that have been constructed, determining the path that needs molecular dynamic calculation according to the feature input file of amber (such as min.in), and saving the path; based on the current environment, writing the environment variables needed at runtime into the calculation call script (named run.sh), and each path will run the amber program; running the npt ensemble dynamic process, and obtaining the system equilibrium structure; determining the parallelism according to the number of free CPUs in the computing node, and running the calculations in queue according to the path list; if the allocation node contains gpu cards, the number of tasks running at the same time is determined according to the number of idle gpu cards, and each task is randomly allocated to each gpu card.

step B: after the above steps are completed, copying the task execution scripts to the directory, including scripts used by the slurm scheduling system, scripts used to construct remd input generation programs and process data; in order to run the scheduling module, tasks need to be allocated to computing nodes with 4 or 6 GPU cards; according to the total number of lambda, generating the corresponding group file, calling the parallel version of the amber program, and running the replica exchange dynamics based on the Hamiltonian; the temperature of each replica is the same, but each replica corresponds to a scale factor (lambda, the value is between 0-1), each lambda is used to affect the interaction between the bonding and non-bonding of the replica, and each replica runs a molecular dynamic simulation of a predetermined number of steps; according to the metropolis standard, each adjacent replica configuration can be exchanged to complete sampling in phase space; this step requires a longer running time, which is usually several hours; after the completion, copying the trajectory file to the corresponding lambda value folder, generating the script for subsequent data processing, and finally generating the handle file to mark the calculation state.

step C: generating a new slurm scheduling script and processing data in the cpu queue: generating input files of cpptraj, using the modified cpptraj program to parse the mdcrd trajectory file, and creating a new process to parse the trajectory in each folder file to achieve the effect of multi-process operation; after parsing, a large number of trajectory files in crd format are obtained, which are combined with each prmtop file to generate a new amber calculation input file; the number of files is the number of images multiplied by the number of pre-extracted trajectories; combining these files into a new calculation queue for single point energy calculation of molecular dynamics; according to the number of free CPU cores, the maximum number of simultaneous computing tasks is determined; when a certain task ends, new tasks are sequentially extracted from the queue and assigned to a new process for calculation, which makes full use of the multi-core environment; removing the handle file from the previous step;

step D: using regular expressions to extract the energy value in the log file, and writing it to the corresponding energy-*.dat file; cleaning up the extra files, and bringing the data into the free energy calculation formula to complete the calculation process;

wherein the calculation amount of the step B is very large and needs to be completed on the gpu, and a series of compilation level optimizations are made to the amber program according to the cpu/gpu model of the cluster.

The invention adopting the above technical solutions has the following advantages:

(1) Strong fault tolerance, the failure of a single task will not affect other tasks. According to the previous technical process, once an error occurs at a certain point during runtime, it is easy to cause the entire calculation process to fail. Although it can be repaired, it will bring extra human work or more code refactoring. Moreover, in practice, due to the instability of the cluster system, occasional unknown errors (such as insufficient hard disk space, io exceptions, etc.) often lead to the failure of the entire process, which is a limiting factor.

(2) Parallel scheduling calculations to maximize the use of gpu and cpu clusters; the original calculation workflow is close to serial use, and the cluster multi-core environment cannot be conveniently used, which leads to a waste of cores and time-consuming; the track file parsing process using a third-party python library makes the data parsing speed very slow. The balance calculation part of the molecular dynamic program often requires a long simulation time (a dozen nanoseconds). The GPU is used to accelerate the calculation; after the calculation is completed, the subsequent data processing module is used to obtain the required data results. As the calculation queue is not distinguished at runtime, the machine-time cost will be more.

(3) The use of hard disks is reasonable. When processing a large number of small files, the intermediate files are cleared while parsing, which reduces the peak value of hard disk usage, so that the result analysis of multiple molecules can be run at the same time in a limited node.

The invention brings the following effects:

(1) The calculation configuration is flexible. Non-professionals only need to write configuration documents and generate runtime scripts through configuration. The entire process steps can be automatically executed, and certain steps can also be specified for calculation, which has good calculation decoupling.

(2) The balance calculation of molecular dynamics makes full use of the single machine with multiple GPU cards; mass data analysis is performed on the cpu side and makes full use of the multi-core processing architecture.

(3) Using statistical data and the characteristics of the lisp language itself, with a small amount of code to solve the situation that some individual errors at runtime will cause the failure of the overall calculation process.

(4) The high-speed trajectory analysis module can extract tens of thousands of trajectories within a few minutes on a multi-core machine; while this part may take 5-6 hours in the past to generate new molecular dynamics input files (used by amber).

(5) The computing resources are fully utilized, and the hard disk usage is reduced, especially when multiple tasks are running at the same time; the reasonable use of hard disk makes the high peak of hard disk usage significantly reduced, and the overall cost performance is high.

### BRIEF DESCRIPTION OF THE DRAWINGS

FIG. 1 is the free energy calculation framework of rest2 for a single molecule in the embodiment.

### DESCRIPTION OF THE EMBODIMENTS

The preferred embodiments of the invention will be further described in detail below in conjunction with the drawings:

A free energy perturbation computation scheduling method used in a heterogeneous cluster environment, wherein the specific calculation steps are:

Step1: Configuring the computing environment file (config.lsp) in a key-value manner. The parameters include: the path of each binary program, file directory, queue characteristic parameters, etc.

Step2: Before calculating a batch of molecules, creating a calculation handle file according to actual needs in the directories that need to be calculated such as charge and vdw, and naming it as single-run-unit, and the scheduler controls the follow-up calculation steps based on whether this file exists. The file names in the calculation directory are named with lambda values, that is, in the form of numeric strings, which are sorted according to the size of the string numbers to be consistent with other input templates.

Step3: After the preparation is completed, the scheduler will recursively scan the folders of the current directory, and generate the run.sh file that calls the amber program based on the min.in and template files contained in the sub-directory, and then summarize the execution file paths and run each Run.sh under the path. This step is to perform npt ensemble simulation on the structure to obtain the equilibrium structure at each lambda value. The running time of each run.sh is not long and this step will not take too much time.

Step4: After the script finds the single-run-unit directory, it will copy the task calculation template to this directory, generate the group.dat file and run the script run_gpu.sh according to the current directory file based on the predefined rules, execute run_gpu.sh file; perform long-term remd calculation in the gpu node, and finally get the track file mdcrd. Here, a remd task uses 6 GPU cards for parallel calculation.

Step 5: After tasks running on the gpu is completed, the trajectory file needs to be parsed. The corresponding directory will continue to perform data processing: first of all, generating the input files required by the cpptraj program (customized version for inperd analysis), parsing all the coordinate files, generating amber input file at the same time, and getting ready to calculate the single point energy of each structure.

Step 6: After the above steps are completed, tens of thousands of small files will eventually be generated, which are used to calculate a single point of energy for amber. Serial calculation is time-consuming, so we use the classic producer-consumer model to queue up computing tasks to make full use of the CPU multi-core resources in the node. At the same time, the calculation and analysis tasks generated by each molecule are summarized in a data processing task, which facilitates the use of the cluster scheduling system and the subsequent error debugging.

Step 7: Using regular expressions to extract the required data from the log file and write it to the corresponding energy-*.dat file. At the same time, deleting the intermediate file to reduce the usage of the hard disk. After the analysis is completed, checking the data integrity, and saving and cleaning up the file to end the calculation process.

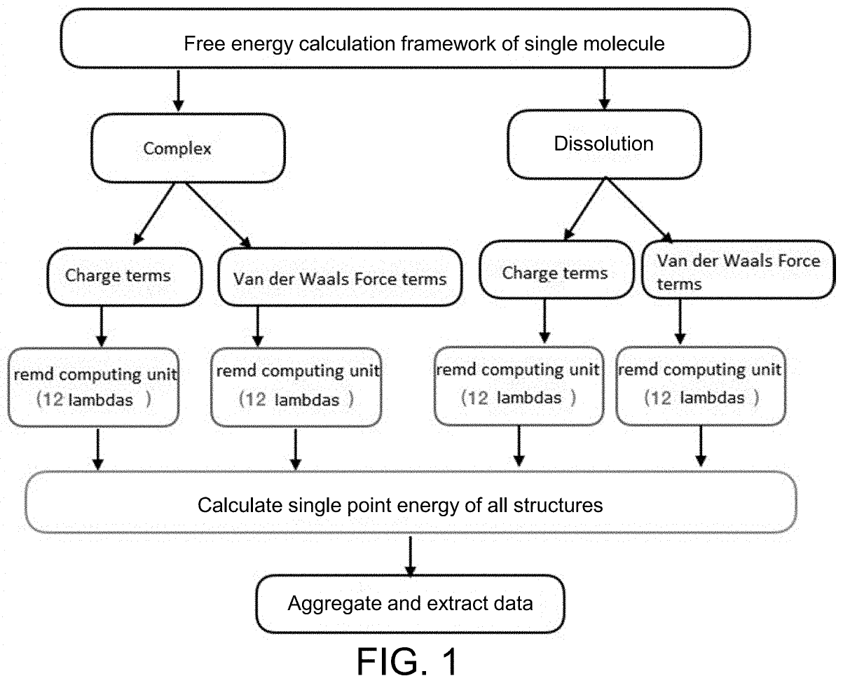

As shown in FIG. 1: remd calculation, wherein the script automatically generates the input files needed for the calculation, performs the gpu calculation, and starts the subsequent analytical calculation process after the calculation is completed.

As shown in FIG. 1: it is mainly the recalculation and energy analysis part, which is carried out in the cpu queue. Because the recalculation process requires a large number of independent calculation tasks and generates a large number of intermediate files, the most conventional producer-consumer parallel calculation is adopted. During the process of parsing the log file, the previous intermediate files will be deleted to reduce the use of the hard disk, and the pmemd.mpi.CUDA program runs.

The above content is a further detailed description in combination with specific preferred embodiments of this invention, and it cannot be considered that the specific implementation of this invention is limited to the description. For those of ordinary skill in the technical field to which this invention belongs, a number of simple deductions or substitutions can be made without departing from the concept of this invention, which should be regarded as belonging to the protection scope of this invention.

1. A free energy perturbation computation scheduling method used in a heterogeneous cluster environment, comprising following steps:
step A: performing an npt ensemble dynamics simulation process through a pre-built molecular/protein structure and input files to obtain an equilibrium structure;
step B: running a replica exchange dynamic calculation based on Hamiltonian, and a temperature of each replica is the same, but each replica corresponds to a scale factor namely lambda, with a value between 0-1, each lambda is used to affect bonding and non-bonding interactions of the replica, and each replica runs a molecular dynamic simulation with a predetermined number of steps, according to metropolis standard, a configuration of each adjacent replica is exchanged and sampled in a phase space to complete the replica exchange dynamic calculation;
step C: after completing step B, analyzing a trajectory file and combining the trajectory file with various prmtops to generate a new amber calculation input, and calculating a single point energy corresponding to each trajectory file after combination to calculate a new energy; and
step D: using regular expressions to extract an energy value in a log file, cleaning up intermediate temporary files, and bringing the energy value into a free energy definition formula to obtain a free energy value.
